(19) United States
(12) Patent Application Publication
(10) Pub. No.: US 2013/0150643 A1
(43) Pub. Date: Jun. 13, 2013

(54) MIXED-PHASE OPERATION OF BUTENES METATHESIS PROCESS FOR MAXIMIZING PROPYLENE PRODUCTION

(71) Applicants: Travis Conant, Richmond, TX (US); Scott A. Stevenson, Houston, TX (US)

(72) Inventors: Travis Conant, Richmond, TX (US); Scott A. Stevenson, Houston, TX (US)

(73) Assignee: SAUDI BASIC INDUSTRIES CORPORATION, RIYADH (SA)

(21) Appl. No.: 13/315,058

(22) Filed: Dec. 8, 2011

(51) Int. Cl. C07C 5/00

(52) U.S. Cl. 585/647; 585/643

(57) ABSTRACT

Methods for olefin metathesis including contacting a olefin feed stream with a metathesis catalyst at a temperature and at a pressure sufficient to maintain the reactor olefin compositions in a mixed-phase condition including components in the liquid phase and components in the vapor phase, where the mixed-phase reaction conditions shift the equilibrium to desired product olefins.

### BACKGROUND OF THE INVENTION

1. Field of the Invention

Embodiments of the present invention relate to methods for metathesis catalytic reactions performed under mixed phase conditions.

More particularly, the present invention relates to methods for metathesis catalytic reactions performed under mixed phase conditions, where a rhenium based catalyst supported on alumina is exposed to a normal butenes feed under mixed-phase metathesis reaction conditions evidencing similar catalyst lifetime compared to the same catalyst operated under gas phase metathesis conditions and improved butenes conversion compared to the same catalyst operated under both gas and liquid phase metathesis conditions.

2. Description of the Related Art

Metathesis is a reaction for transforming starting olefins into different olefins by exchanging substituents between olefins. For example, the metathesis of 1-butene and 2-butenes produces propylene and 2-pentene. This reaction was demonstrated by Engelhard and Zsinka in the early 1980's, while the conversion of isobutylene and 2-butenes into propylene and 2-methyl-2-butene was reported by Nakamura, et al., in 1972. The dismutations of 2-pentene to give 2-butenes and hexene and of 1-butene to give ethylene and 3-hexene were demonstrated by Banks in the mid-1960's.

The prior art on metathesis is extensive, yet the prior art of the metathesis of mixed butenes (without the addition of ethylene) is rather sparse. A survey of this prior art reveals no mixed-phase processes to increase the butenes conversion. U.S. Pat. No. 6,777,582 disclosed the process for the metathesis of butenes without ethylene. While there are no patents which specify the metathesis of butenes without ethylene in the liquid phase, U.S. Pat. No. 6,743,958 does describe the liquid phase metathesis of 2-butene and ethylene.

While many patents and pending applications relate to or concern metathesis of olefins, there is still a need in the art for metathesis reactions that produce tailored products from a given olefin under controlled conditions, especially when the conditions and catalysts produce unexpectedly high olefin conversions and desired product distributions.

### SUMMARY OF THE INVENTION

Embodiments of this invention provide systems and methods for olefin metathesis performed under mixed-phase reaction conditions.

Embodiments of this invention provide systems for olefin metathesis including a metathesis reactor including a metathesis catalyst bed, where the reactor includes a feed port and an effluent port. The feed port is adapted to receive an olefin input stream including a starting olefin stream and a plurality of recycle streams. The effluent port is adapted to discharge a crude olefin product stream. The systems also include a fractionation subsystem including multiple columns for the separation of C₂'s (a deethanizer), C₃'s (depropanizer), C₄'s (debutanizer), and C₅'s (depentanizer). The crude product stream is first forwarded to the deethanizer, where an overhead light stream including ethylene is separated and recycled to the metathesis reactor. A bottom deethanizer stream including >C₂'s olefins is forwarded to the depropanizer, where an overhead propylene product stream is produced and a bottom depropanizer stream including >C₃ olefins stream is forwarded to a depentanizer. The depentanizer separates the bottom depropanizer stream into a hexene product stream olefins and an overhead stream including C₄ and C₅ olefins. The C₄ and C₅ olefins stream is forwarded to the debutanizer. The debutanizer separates the C₄ and C₅ olefins stream into an overhead C₄ olefins stream and a bottom C₅ olefins stream, which is recycled back to the metathesis reactor. The systems also include one or more olefin isomerization reactors to isomerize olefin mixtures in the starting, recycle and/or product streams. In certain embodiments, the systems include an olefin isomerization reactor including an olefin isomerization catalyst used to adjust the olefin composition of the olefin starting stream. In other embodiments, the systems include other olefin isomerization reactors used to adjust the olefin compositions of one or more recycle and/or product streams. In other embodiments, the systems include olefin isomerization reactors to isomerize the feedstock and/or one or more recycle and/or product. The systems may also include secondary reactors that convert one or more product olefins into other produces. In the case of butenes metathesis, the systems may include an aromatization reactor for converting hexenes into aromatics such as benzene.

Embodiments of this invention provide methods for olefin metathesis under reaction conditions including a reaction temperature and a reaction pressure, where the reaction pressure is above a dew point pressure of an equilibrium reaction mixture at the reaction temperature.

Embodiments of this invention provide methods for olefin metathesis under reaction conditions that include a reaction temperature and a reaction pressure, where the reaction pressure is above a dew point pressure and below a bubble point pressure of the reaction mixture at the reaction temperature, i.e., the metathesis is under mixed-phase reaction conditions.

Embodiments of this invention provide methods for olefin metathesis under reaction conditions that include a reaction temperature and a reaction pressure, where an equilibrium reaction mixture exists as a two phase system, a liquid phase and a gas phase, where the operating pressure causes heavier components to preferentially condense on the catalyst or in the pores of the catalyst, while the lighter components do not tend to condense, or condense to a lesser extent. In the case of metathesis of butenes, the heavier components include pentenes and hexenes and the lighter components include ethylene and propylene.

Embodiments of this invention provide methods for butenes metathesis using rhenuim based catalysts under mixed-phase reaction conditions. In certain embodiments, the reaction conditions are at a temperature and at a pressure above a dew point pressure of an equilibrium product mixture of starting butenes and product ethylene, propylene, 2-pentene, and 3 -hexene. In other embodiments, the reaction conditions are at a temperature and at a pressure above a dew point of heavier reaction products 2-pentene and/or 3-hexene.

Embodiments of this invention provide methods for olefin metathesis include contacting an olefin feed stream with a metathesis catalyst in a metathesis reactor at a reaction temperature and at a reaction pressure sufficient to maintain an olefin reaction mixture in a mixed-phase condition including components in the liquid phase and components in the gas phase to produce an olefin effluent stream. The methods also include recovering an olefin product from the olefin effluent stream. In certain embodiments, the methods may further include fractionating the effluent stream to form a plurality of fraction streams and recycling at least a portion of at least one of the plurality of fraction streams to the metathesis reactor. The step of recovering an olefin product from the olefin effluent stream comprises recovering an olefin product from at least a portion of at least one of the plurality of fraction streams. In other embodiments, the methods may further include directing at least a portion of at least one of the plurality of fraction streams to an olefin isomerization reactor and therein subjecting the same to olefin isomerization reaction. In other embodiments, the methods may further include directing at least a portion of at least one of the plurality of fraction streams to an aromatization reactor and therein subjecting the same to an aromitzation reaction. In other embodiments, the methods may further include directing the olefin feed stream to an olefin isomerization reactor and therein subjecting the same to an olefin isomerization reaction prior to the step of contacting.

In other embodiments, the reaction pressure is a pressure above a dew point pressure of the reaction mixture at the reaction temperature. In other embodiments, the reaction pressure is above a dew point pressure and below a bubble point pressure of the reaction mixture at the reaction temperature. In other embodiments, the reaction temperature and the reaction pressure are selected such that the olefin reaction mixture exists as a two phase system, a liquid phase and a gas phase, where at least one heavier olefin condenses and the lighter olefins do not condense, where the heavier olefins are olefins in the olefin reaction mixture having higher boiling point temperatures, lowest dew point temperatures, than the lighter olefins, which have lower boiling points in the olefin reaction mixture. In other embodiments, the reaction pressure is above a dew point pressure of at least one of the heavier olefins in the olefin reaction mixture at the reaction temperature.

In other embodiments, the reaction temperature is between about 40° and about 100° C. and the reaction pressure is sufficient to maintain the olefin reaction mixture in a mixed-phase region of the olefin reaction mixture phase diagram. In other embodiments, the reaction temperature is between about 40° and about 90° C. and the reaction pressure is sufficient to maintain the olefin reaction mixture in a mixed-phase region of the olefin reaction mixture phase diagram. In other embodiments, the reaction temperature is between about 40° and about 80° C. and the reaction pressure is sufficient to maintain the olefin reaction mixture in a mixed-phase region of the olefin reaction mixture phase diagram. In other embodiments, the reaction temperature is between about 40° and about 60° C. and the reaction pressure is sufficient to maintain the olefin reaction mixture in a mixed-phase region of the olefin reaction mixture phase diagram.

In other embodiments, the starting olefin stream comprises a mixture of C₂ to C₁₂ olefins. In other embodiments, the starting olefin stream comprises ethylene and butenes. In other embodiments, the starting olefin stream comprises ethylene and pentenes. In other embodiments, the starting olefin stream is a butenes stream including 1-butene and 2-butenes. In other embodiments, the reaction pressure is above a dew point pressure of the reaction mixture including ethylene, propylene, normal butenes, 2-pentene, and 3-hexene. In other embodiments, the reaction pressure is above a dew point of at least 3-hexene.

In other embodiments, the metathesis catalyst comprises a catalyst capable of olefin metathesis under conditions. In other embodiments, the metathesis catalyst is selected from the group consisting of supported and unsupported molybdenum metathesis catalysts, tungsten metathesis catalysts, rhenium metathesis catalysts, niobium metathesis catalysts, tantalum metathesis catalysts, tellurium metathesis catalysts, and mixtures or combinations thereof. In other embodiments, the metathesis catalyst is selected from the group consisting of supported molybdenum metathesis catalysts, supported tungsten metathesis catalysts, supported rhenium metathesis catalysts, supported niobium metathesis catalysts, supported tantalum metathesis catalysts, supported tellurium oxide metathesis catalysts, supported molybdenum and tungsten sulfide metathesis catalysts, supported molybdenum and tungsten hexacarbonyl metathesis catalysts, and mixtures or combinations thereof. In other embodiments, the metathesis catalyst comprises a rhenium metathesis catalyst. In other embodiments, the metathesis catalyst comprises supported rhenium metathesis catalysts. In other embodiments, the supported rhenium metathesis catalyst comprises Re₂O₇ and Al₂O₃.

### BRIEF DESCRIPTION OF THE DRAWINGS

The invention can be better understood with reference to the following detailed description together with the appended illustrative drawings in which like elements are numbered the same:

FIG. 1 depicts a schematic of an embodiment of an apparatus for carrying out the process of the invention.

FIG. 2 depicts a comparison of catalyst longevity for gas and liquid phase experiments at 50° C. for a 5 wt % Re₂O₇/Al₂O₃ catalyst.

FIG. 3 depicts a comparison of the butenes conversion in the gas and liquid phase experiments with the calculated equilibrium conversion values for each phase (calculated using Aspen software).

FIG. 4 depicts calculated dew point and-bubble point curves of a calculated metathesis equilibrium product distribution. (Equilibrium and dew/bubble point data calculated using Aspen software.)

FIG. 5 depicts a comparison of catalyst longevity for mixed-phase and liquid phase experiments at 50° C. for a 5wt % Re₂O₇/Al₂O₃ catalyst.

FIG. 6 depicts a comparison of the butenes conversion for mixed-phase and liquid phase experiments with the calculated equilibrium conversion values for each phase (calculated using Aspen software).

FIG. 7 depicts plot of conversion versus time on stream for gas phase metathesis and mixed-phase metathesis at 75° C. at pressures above (90 psig; mixed-phase) and below (40 psig; gas phase) the calculated dew point pressure, respectively.

FIG. 8 depicts plot of conversion versus time on stream for a Re₂O₂/Al₂O₃ catalyst which has undergone two consecutive in-situ regeneration cycles in air at 500° C.

### DEFINITIONS USED IN THE INVENTION

The term “dew point” means the temperature at which a gaseous metathesis reaction mixture begins to condense at a given pressure. Alternatively, the dew point is the pressure at which a gaseous metathesis reaction mixture begins to condense at a given temperature.

The term “bubble point” means the temperature at which a liquid metathesis reaction mixture begins to boil at a given pressure. Alternatively, the bubble point is the pressure at which a liquid metathesis reaction mixture begins to boil at a given temperature.

The term “C₂'s” means a mixture of hydrocarbons having 2 carbon atoms.

The term “C₃'s” means a mixture of hydrocarbons having 3 carbon atoms.

The term “C₄'s” means a mixture of hydrocarbons having 4 carbon atoms.

The term “C₅'s” means a mixture of hydrocarbons having 5 carbon atoms.

The term “C₆'s” means a mixture of hydrocarbons having 2 carbon atoms.

The term “deethanizer” means a column that is designed to remove C₂'s from a mixture containing hydrocarbons including three or more carbon atoms.

The term “depropanizer” means a column that is designed to remove C₃'s from a mixture containing hydrocarbons including four or more carbon atoms.

The term “debutanizer” means a column that is designed to remove C₄'s from a mixture containing hydrocarbons including five or more carbon atoms.

The term “depentanizer” means a column that is designed to remove C₅'s from a mixture containing hydrocarbons including six or more carbon atoms.

The term “>C₂'s olefins” means olefins having primarily three or more carbon atoms, i.e., the olefins may include minor amounts of C₂ olefins.

The term “>C₃ olefins” means olefins primarily having four or more carbon atoms, i.e., the olefins may include minor amounts of C₂ and C₃ olefins.

The term “C₄ and C₅ olefins” means olefins having primarily four and five carbon atoms, i.e., the olefins may include minor amounts of C₂, C₃ and C₆ olefins.

### DETAILED DESCRIPTION OF THE INVENTION

The inventor has found that low-temperature metathesis conversion using a rhenium metathesis catalyst such as Re₂O₇/Al₂O₃ can be increased, thereby increasing propylene productivity, by operating the metathesis reaction under mixed-phase reaction conditions. In one embodiment, the process involves the metathesis of mixed butenes (1-butene and 2-butenes) into a product mixture including ethylene, propylene, un-reacted mixed butenes, pentenes, and hexenes. Experiments in the gas and liquid phases described herein have shown that equilibrium calculations accurately predict the expected butenes conversion and product selectivities. The metathesis conversion value was found to be approximately 62% for both gas and liquid phase experiments. The inventor has found that by operating above a dew point pressure at a given temperature (i.e., 50° C. and 75 ° C.), but below a bubble point pressure, the butenes conversion may be increased by an amount between about 2% and 10%. In the case of butenes metathesis at 50° C. and 40 psig compared to a liquid phase reaction at the same temperature, the conversion was increased by amount of about 5% from about 62% to about 67%. In the case of butenes metathesis at 75 ° C. and 90 psig compared to a gas phase reaction at the same temperature, the conversion was also increased by amount of about 5% from about 62% to about 67%. In certain embodiments the reaction is operated at a pressure just below a dew point curve of an equilibrium product mixture. It is thought that operating at this pressure causes heavier products, such as pentenes and hexenes in the case of the metathesis of butenes, to preferentially condense on the catalyst or in the pores of the catalyst, while the lighter components, such as ethylene and propylene in the case of metathesis of butenes, do not tend to condense, or condense to a lesser extent. The condensation of heavier products is thought to induce a driving force for the effective removal of lighter components from the catalyst pores, resulting in a shift in the reaction equilibrium increasing butene conversion.

Embodiments of this invention broadly relate to systems for olefin metathesis including a metathesis reactor including a metathesis catalyst bed, where the reactor includes a feed port and an effluent port. The feed port is adapted to receive an olefin input stream including a starting olefin stream and a plurality of recycle streams. The effluent port is adapted to discharge a crude olefin product stream. The systems also include a fractionation subsystem including multiple columns for the separation of C₂'s (a deethanizer), C₃'s (depropanizer), C₄'s (debutanizer), and C₅'s (depentanizer). The crude product stream is first forwarded to the deethanizer, where an overhead light stream including ethylene is separated and recycled to the metathesis reactor. A bottom deethanizer stream including >C₂'s olefins is forwarded to the depropanizer, where an overhead propylene product stream is produced and a bottom depropanizer stream including >C₃ olefins stream is forwarded to a depentanizer. The depentanizer separates the bottom depropanizer stream into a hexene product stream olefins and an overhead stream including C₄ and C₅ olefins. The C₄ and C₅ olefins stream is forwarded to the debutanizer. The debutanizer separates the C₄ and C₅ olefins stream into an overhead C₄ olefins stream and a bottom C₅ olefins stream, which is recycled back to the metathesis reactor. The systems also include one or more olefin isomerization reactors to isomerize olefin mixtures in the starting, recycle and/or product streams. In certain embodiments, the systems include an olefin isomerization reactor used to adjust the olefin composition of the olefin starting stream. In other embodiments, the systems include an olefin isomerization reactor used to adjust the olefin composition of one or more recycle streams. In other embodiments, the systems may also include both olefin isomerization reactors. The systems may also include secondary reactors that convert one or more product olefins into other produces. In the case of butenes metathesis, the systems may also include an aromatization reactor for converting product hexene olefins into benzene.

Embodiments of this invention broadly relate to methods and systems for olefin metathesis performed under mixed-phase reaction conditions. In certain embodiments, the reaction conditions include a reaction temperature and a reaction pressure, where the reaction pressure is above a dew point pressure of an equilibrium reaction mixture at the reaction temperature. In certain embodiments, the reaction conditions include a reaction temperature and a reaction pressure, where the reaction pressure is above a dew point pressure of an equilibrium reaction mixture by at least 1% of the dew point pressure at the reaction temperature; provided the reaction pressure is below the bubble point pressure of the equilibrium reaction mixture at the reaction temperature. In certain embodiments, the reaction conditions include a reaction temperature and a reaction pressure, where the reaction pressure is above a dew point pressure of an equilibrium reaction mixture by at least 2.5% of the dew point pressure at the reaction temperature; provided the reaction pressure is below the bubble point pressure of the equilibrium reaction mixture at the reaction temperature. In certain embodiments, the reaction conditions include a reaction temperature and a reaction pressure, where the reaction pressure is above a dew point pressure of an equilibrium reaction mixture by at least 5% of the dew point pressure at the reaction temperature; provided the reaction pressure is below the bubble point pressure of the equilibrium reaction mixture at the reaction temperature. In certain embodiments, the reaction conditions include a reaction temperature and a reaction pressure, where the reaction pressure is above a dew point pressure of an equilibrium reaction mixture by at least 10% of the dew point pressure at the reaction temperature; provided the reaction pressure is below the bubble point pressure of the equilibrium reaction mixture at the reaction temperature. In other embodiments, the reaction conditions include a reaction temperature and a reaction pressure, where the reaction pressure is above a dew point pressure and below a bubble point pressure of an equilibrium reaction mixture at the reaction temperature. In other embodiments, the reaction conditions include a reaction temperature and a reaction pressure, where an equilibrium reaction mixture exists as a two phase system, a liquid phase and a gas phase, where at least one heavier olefin condenses and lighter olefins do not condense. In other embodiments, the reaction conditions include a reaction temperature and a reaction pressure, where the equilibrium reaction mixture exists as a two phase system, a liquid phase and a gas phase, where heavier olefins condense and lighter olefins do not condense. In other embodiments, the reaction conditions include a reaction temperature and a reaction pressure, where the reaction pressure is above a dew point pressure of at least one heavier olefins in the product mixture at the reaction temperature. The term “heavier olefins” means olefins having higher boiling point temperatures, lowest dew point temperatures, than “lighter olefins”, which have lower boiling point temperatures in the reaction mixture. Of course, the exact composition of the heavier olefins and lighter olefins in a given metathesis reaction will dependent on the starting olefins. In certain embodiments, the temperature is between about 40° and about 100° C. and the pressure is sufficient to maintain the reaction conditions in a mixed-phase region of the equilibrium metathesis composition phase diagram. In certain embodiments, the temperature is between about 40° and about 90° C. and the pressure is sufficient to maintain the reaction conditions in a mixed-phase region of the equilibrium metathesis composition phase diagram. In certain embodiments, the temperature is between about 40° and about 80° C. and the pressure is sufficient to maintain the reaction conditions in a mixed-phase region of the equilibrium metathesis composition phase diagram. In certain embodiments, the temperature is between about 40° and about 60° C. and the pressure is sufficient to maintain the reaction conditions in a mixed- phase region of the equilibrium metathesis composition phase diagram. In other embodiments, the reaction temperature is about 50° C. and the pressure is about 40 psig. In other embodiments, the reaction temperature is about 75° C. and the pressure is about 90 psig.

Mixed butenes are viewed as a low cost feedstock that is readily available as by-products from crackers. The conversion of a low cost feedstock into higher valued products such as propylene and hexenes from butenes makes the metathesis processes of increased interest in the industry as cracker feeds transition to heavier cuts.

Based on earlier work and on a knowledge of prior catalyst art, the inventor investigated the differences between the gas and liquid phase metathesis reactions in an attempt to discover a different and potentially improved method for the metathesis of starting lower value olefins into higher value olefins. It was found that operating the metathesis reaction under mixed-phase conditions results in two benefits: (1) increased catalyst lifetime over the liquid phase and (2) increased butenes conversion over the gas and liquid phases. By mixed-phase conditions, the inventors mean that an operating pressure at a given temperature above a dew point pressure of the equilibrium product distribution.

Furthermore, experiments showed that the rhenium-based catalysts may undergo a number of regenerations in air at 500° C.; regaining 100% of their original activity after being completely deactivated.

### Suitable Reagents and Catalyst

Suitable olefin feedstock stream for use in the methods and systems of this invention include, without limitation, mixtures of C₂ to C₁₂ olefins. In certain embodiments, the mixture of olefins comprises ethylene and butenes. In other embodiments, the mixture of olefins comprises butenes including 1-butene and 2-butenes. In other embodiments, the mixture of olefins comprises ethylene and pentenes.

Suitable metathesis catalyst for use in the methods and systems of this invention include, without limitation, any catalyst known in the art of olefin metathesis including supported and unsupported molybdenum metathesis catalysts, tungsten metathesis catalysts, rhenium metathesis catalysts, niobium metathesis catalysts, tantalum metathesis catalysts, tellurium metathesis catalysts, and mixtures or combinations thereof. Exemplary examples of suitable metathesis catalysts include, without limitation, supported molybdenum metathesis catalysts, supported tungsten metathesis catalysts, supported rhenium metathesis catalysts, supported niobium metathesis catalysts, supported tantalum metathesis catalysts, supported tellurium oxide metathesis catalysts, supported molybdenum and tungsten sulfide metathesis catalysts, supported molybdenum and tungsten hexacarbonyl metathesis catalysts, any other catalyst capable of metathesizing olefins, and mixtures or combinations thereof. In certain embodiment, the metathesis catalyst are rhenium metathesis catalysts. In other embodiments, the metathesis catalysts are supported rhenium metathesis catalysts.

### Systems of the Invention

Introduction

The conversion of mixed butenes to propylene and hexenes is economically very attractive, because butenes are low value cracker by-products. Mixed butenes are currently produced as by-products from steam cracker units and may be purposely produced from other types of cracker units as well. As heavier feeds are cracked, it is expected that the amount of butenes available for use in the metathesis process of this invention will increase in the future.

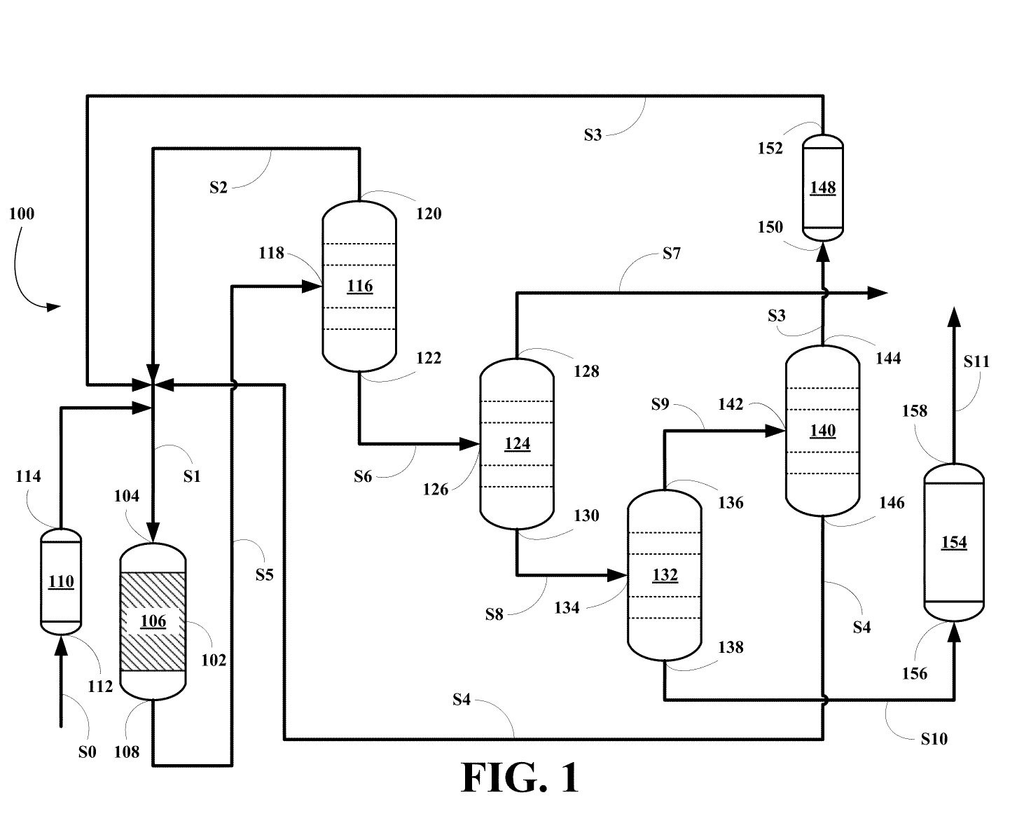

The key catalytic process in the system of this invention is a metathesis reaction carried out in a metathesis reactor, where starting low value olefins such as butenes are converted to higher value olefins such as a mixture of C₂, C₃, C₄, C₅ and C₆ olefins. The propylene and the hexenes are then separated from the effluent. The hexenes may be subsequently converted to aromatics such as benzene in an aromatization reactor. The remaining olefins are then recycled until the butenes are fully converted into product olefins or are converted below some set point concentration. The entire process is envisioned schematically in FIG. 1.

Referring now to FIG. 1,, an embodiment of a system for the metathesis of butenes, generally 100, is shown to include a metathesis reactor 102 having an inlet or feed port 104, a catalyst zone 106, and an outlet port 108. A metathesis catalyst is contained in the catalyst zone 106 of the metathesis reactor 102. A feed stream S1 enters the reactor 102 through the feed port 104. The feed stream S1 comprises a butenes feedstock stream S0, an ethylene recycle stream S2, a butenes recycles stream S3 and a pentenes recycle stream S4. A crude olefin product stream S5 is withdrawn from the metathesis reactor 102 through the outlet port 108. In certain embodiments, the butenes feedstock stream S0 may be isomerized in an optional first isomerization reactor 110, where the stream S0 enters the isomerization reactor 110 through an inlet port 112 and exits from an outlet port 114. The isomerization reactor 110 is designed to adjust a relative mole ratio of 1-butene to 2-butenes in the feedstock stream S0.

After exiting the metathesis reactor 102, the product stream S5 is then introduced into a deethanizer 116 through an inlet port 118. The deethanizer 116 separates the product stream S5 into the ethylene recycle stream S2, which exits the deethanizer 116 through a first outlet port 120 and a heavies stream S6 including >C₂ olefins, which exits the deethanizer 116 through a second outlet port 122.

The >C₂ olefins stream S6 is introduced into a depropanizer 124 through an inlet port 126. The depropanizer 124 separates the >C₂ olefins stream into a propylene product stream S7, which exits the depropanizer 124 through a first outlet port 128 and a heavies stream S8 including >C₃ olefins, which exits the depropanizer 124 through a second outlet port 130.

The >C₃ olefins stream S8 is introduced into a depentanizer 132 through an inlet port 134. The depentanizer 132 separates the >C₃ olefins stream into a C₄ and C₅ olefins stream S9, which exits the depentanizer 132 through a first outlet port 136 and a hexenes product stream S10, which exits the depentanizer 132 through a second outlet port 138.

The C₄ and C₅ olefins stream S9 is introduced into a debutanizer 140 through an inlet port 142. The debutanizer 140 separates the C₄ and C₅ olefins stream into the butenes recycle stream S3, which exits the debutanizer 140 through a first outlet port 144 and the pentenes recycle stream S4, which exits the debutanizer 140 through a second outlet port 146. In certain embodiments, the butenes recycle stream S3 may be isomerized in an optional second isomerization reactor 148, where the stream S3 enters the isomerization reactor 148 through an inlet port 150 and exits from an outlet port 152 and where the relative mole ratio of 1-butene to 2-butenes is adjusted.

In other embodiments, the system 100 may include both olefin isomerization reactors 110 and 148. In all cases where isomerization reactors are used, the isomerization reactors are designed to change the relative mole ratio of 1-butene to 2-butenes.

In the system of this embodiment, the deethanizer, the depropanizer, the depentanizer and the debutanizer comprise a fractionation subsystem designed to separate the crude olefin product stream into appropriate recycle and product streams. It should be recognized that one of ordinary skill in the art could use a different column configuration; provided that the fractionation produces the appropriate recycle and product streams.

The system 100 may also include an optional aromatization reactor 154. If present, the hexenes stream S10 is introduced into a feed port 156 of the aromatization reactor 154 and an aromatics product stream S11 is withdrawn from an outlet port 158.

The butene feedstock stream S0 may be obtained from one source or a plurality of sources. The relative amounts of 1-butene and 2-butenes in the butenes feedstock stream S0 may vary depending on the source or sources. If the amount of 1-butene in the feedstock stream S0 is low, then the butene feedstock stream S0 is passed through the double-bond isomerization reactor 110 operating at a temperature greater than 400° C. to convert a portion of the 2-butenes into 1-butene; however, if the amount of 1-butene is relatively high (as might be the case if the butene feedstock was obtained from a steam cracker unit), then the fresh feed may bypass the optional isomerization reactor 110.

The metathesis reactor 102 converts the butenes in the stream S1 into other olefins. As discussed below, the metathesis reaction interconverts olefins such as 1-butene and 2-butenes into olefin products including ethylene, propylene, 2-pentene, and 3-hexene. The extent of conversion of the butenes in the stream S1 depends on the catalyst and reaction conditions, but the overall product distribution for a given feedstock is dictated by thermodynamics and a feed ratio of 1-butene to 2-butenes in the stream S1.

### EXAMPLES OF THE INVENTION

The overall stoichiometry of the metathesis reaction is:
3C₄H₈→2C₃H₆+C₆H₁₂

On a weight basis, the products may contain 50% propylene and 50% 3-hexene. The 50 wt % of hexenes may then be transformed into 46% benzene and 4% hydrogen based on the initial butene feed. This assumes that there are no losses or non-selective reactions. This assumption is not completely true; however, as it is anticipated that the primary reaction byproducts may include cracker products, coke, and small amounts of C₇ materials. Losses may also occur in the separations or recycle purge; the extent of these losses may depend on the amount of unsaturated C₄' components present in the feed.

Olefin Metathesis

The olefin metathesis reaction, which was first reported by Banks and Bailey of Philips Petroleum in 1964, is a reversible rearrangement of olefins by cleavage and reformation of carbon-carbon double bonds. In this reaction, two olefins react followed by reorganization of the double bonds to form two reorganized olefins the substituents R¹⁻⁴ are exchanged, i.e.:

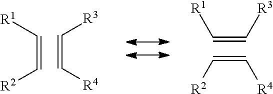

This reaction is widely used for the interconversion of a variety of olefins, and is commercially practiced in the so-called Tri Olefin process, in which ethylene and 2-butenes are converted to propylene. A number of different catalysts are active for this reaction; the early work of the Philips group reported data on supported molybdenum, tungsten, rhenium, niobium, tantalum, and tellurium oxides, molybdenum and tungsten sulfides, and molybdenum and tungsten hexacarbonyls. A wide variety of homogeneous catalysts have also been developed, many of which show very high activities and the ability to metathesize hindered or less-reactive olefins.

The reaction is widely believed to proceed through a four-center intermediate. One important feature of the metathesis reaction is that it merely interchanges molecular fragments among olefins, but cannot by itself change the size of these fragments. In the present reaction, 1-butene may be considered as a combination of a one-carbon fragment and a three-carbon fragment; likewise, 2-butenes may be considered a combination of 2 two-carbon fragments. Metathesis may produce any combination of fragments of butenes including ethylene (1+1), propylene (1+2), 2-pentene (2+3) and 3-hexene (3+3), but metathesis cannot result in any molecule such as 2-hexene or 3-heptene that contains a four carbon fragment. If other reactions, such as double-bond shift isomerization, cracking, and dimerization, are avoided, no olefins larger than C₆ are produced.

In one embodiment of the present invention, the goal of the metathesis reaction is to convert butenes to propylene and 3-hexene. This metathesis reaction requires a mole ratio of 1-butene to 2-butenes of 2:1, because in butenes metathesis, two moles of 1-butene are converted for each mole of 2-butenes converted. If the feed mixture contains less than a 2:1 ratio of 1-butene to 2-butenes, as is likely for many feed and/or recycle streams, the 1-butene to 2-butenes mole ratio will generally need to be adjust especially after metathesis, where the metathesis reaction results in a decrease the 1-butene to 2-butenes mole ratio. Thus, some of the 2-butenes in the recycle stream will generally need to be converted via isomerization to 1-butene.

Olefin Isomerization

As discussed above, the net stoichiometry requires conversion of two molecules of 1-butene for each molecule of 2-butenes consumed. Because of this, it will probably be necessary to convert some of the recycled 2-butenes to 1-butene. Depending on the content of 1-butene in the feed, it may also be desirable to isomerize some of the 2-butenes in the feed as well. This interconversion, or double-bond shift isomerization, is a relatively facile reaction. Active catalysts include alkali or alkaline earth metal oxides, especially magnesium oxide, noble metals, and zeolites. Equilibrium, is the major limitation to 2-butenes isomerization. Although equilibrium is independent of pressure, it is dependent on temperature, with higher temperatures favoring 1-butene.

Operation of the isomerization reactor must, therefore, be at as high a temperature as is possible while still avoiding losses to coking, dimerization, and cracking.

Comparison of Gas and Liquid Phase at 50° C.

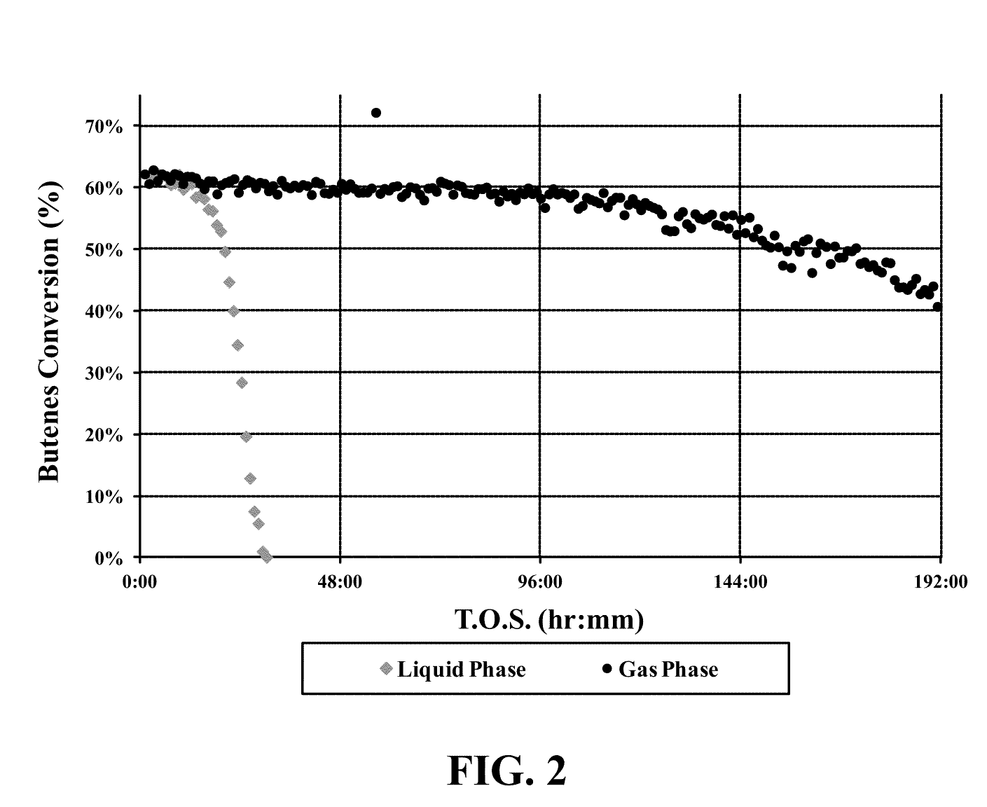

We investigated the differences between metathesis reactions operated under gas phase conditions and liquid phase conditions at a temperature of 50° C. FIG. 2 illustrates the differences between experiments performed in the two different phases. Each experiment was run at an identical weight hourly space velocity (WHSV) of 1.5 hr⁻¹, a 1-butene:2-butene feed ratio of 2:1 and a temperature of 50° C. The only difference between the two experiments was the operating pressure, and consequently the phase. The gas phase reaction was operated at 10 psig, while the liquid phase reaction was operated at 400 psig.

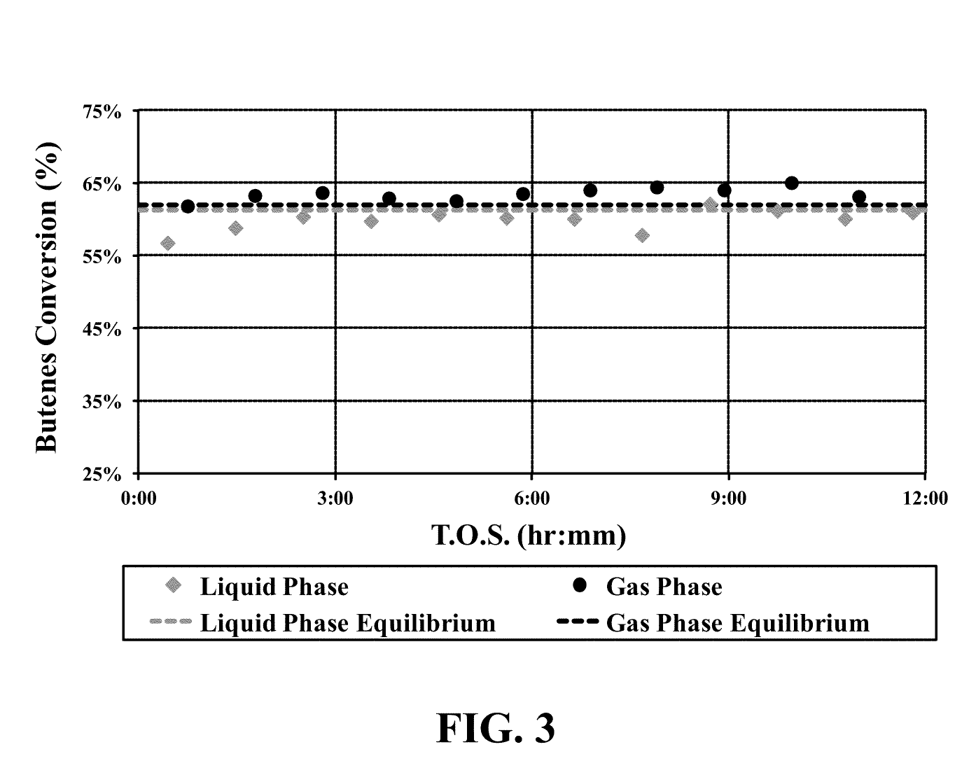

FIG. 2 illustrates that the gas phase reaction resulted in a catalyst lifetime that was nearly 10 times longer than the liquid phase reaction. Additionally, the initial total butenes conversion fits very well with calculated equilibrium values. The calculated equilibrium butenes conversion was calculated for both the gas phase and liquid phase reactions. These values are displayed in FIG. 3. It can be seen that the predicted difference due to operating phase resulted in only a fraction of a percent lower conversion (e.g., from 62.2% down to 61.3%), which is within experimental error of a given run.

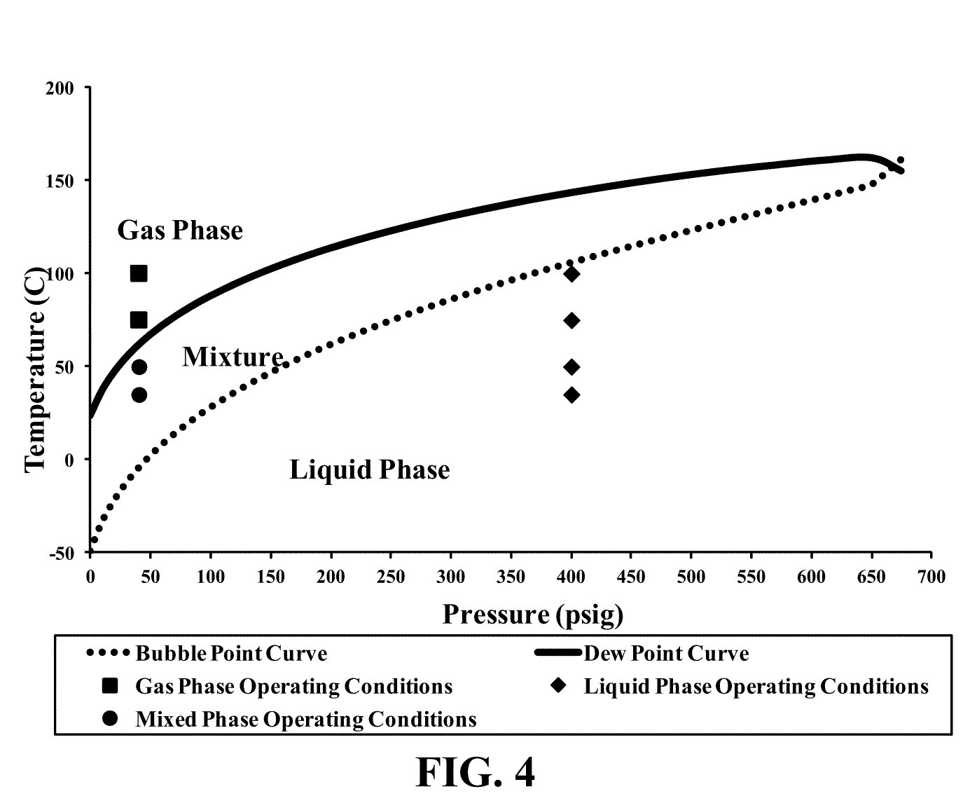

The dew point and bubble point curves were calculated for the equilibrium product mixture using Aspen software in order to determine the experimental conditions for mixed-phase operation. The resulting dew and bubble curves are illustrated in FIG. 4.

Comparison of Mixed and Liquid Phase at 50° C.

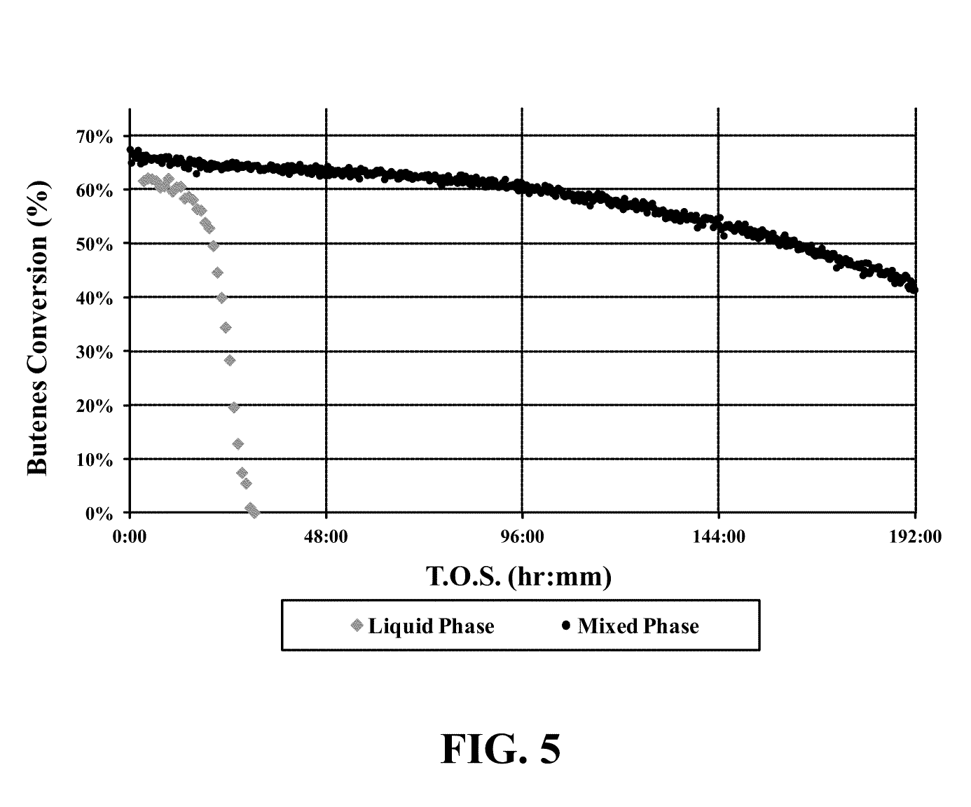

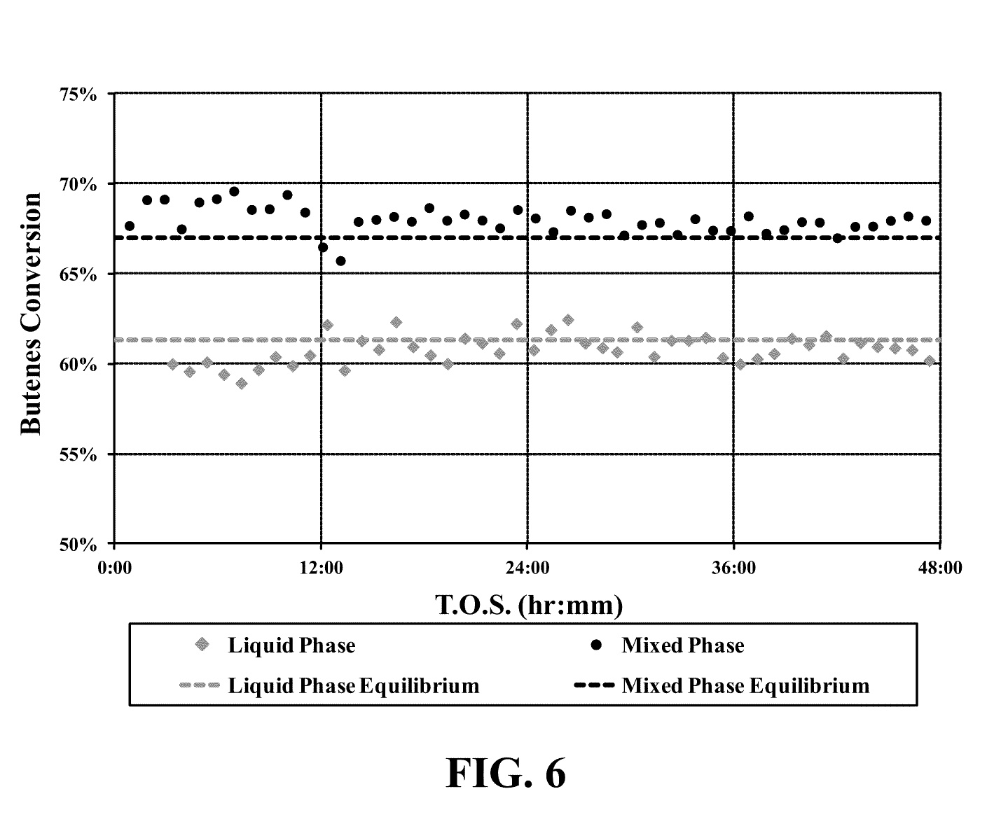

We investigated the differences between metathesis reactions operated under mixed-phase conditions and liquid phase conditions at a temperature of 50° C. FIG. 5 illustrates the differences between experiments performed at the two different conditions. Each experiment was run at an identical weight hourly space velocity (WHSV) of 1.5 hr⁻¹, a 1-butene:2-butene feed ratio of 2:1 and a temperature of 50° C. The only difference between the two experiments was the operating pressure, and consequently the phase. The liquid phase reaction was operated at 400 psig, while the mixed-phase reaction was operated at 40 psig. Two important differences are evident from the data, which are illustrated in FIGS. 2 and 3 and FIGS. 5 and 6: (1) the mixed-phase reaction resulted in a catalyst lifetime that was nearly 10 times longer than the liquid phase reaction and (2) the mixed-phase reaction showed a butenes conversion higher than the liquid phase reaction.

Comparison of Mixed and Gas Phase at 75° C.

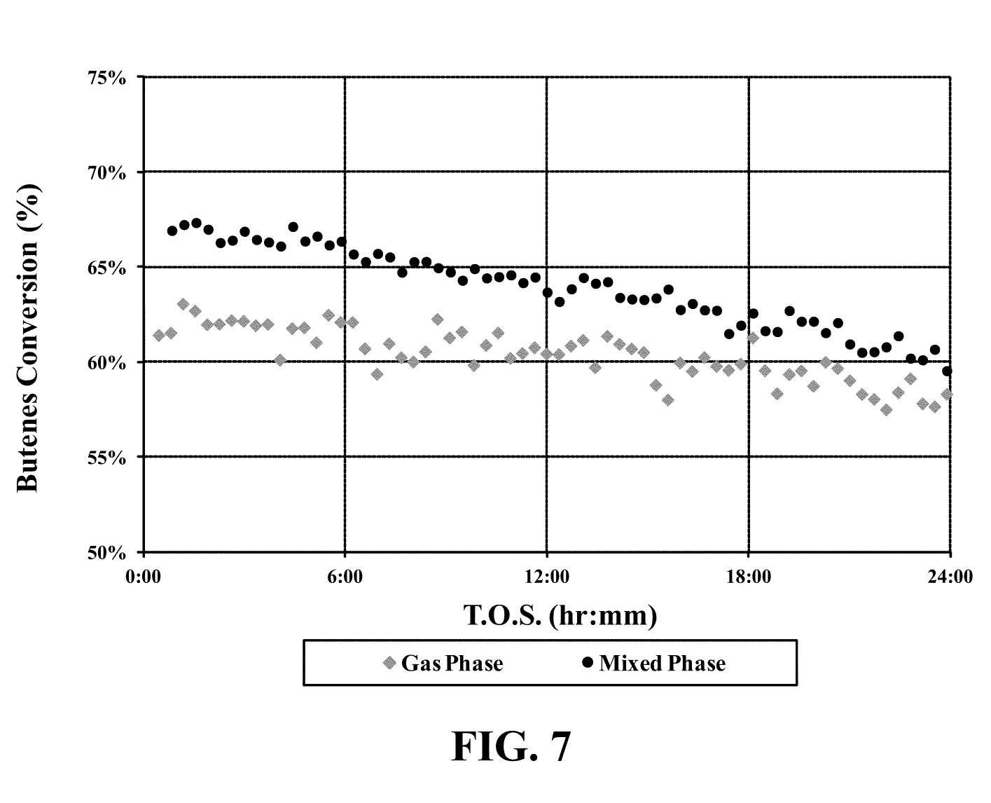

We also investigated the differences between metathesis reactions operated under mixed-phase conditions and gas phase conditions at a temperature of 75° C. FIG. 7 illustrates the observed differences. Each experiment was run at an identical weight hourly space velocity (WHSV) of 1.5 hr⁻¹, a 1-butene:2-butene feed ratio of 2:1 and a temperature of 75° C. The gas phase reaction was operated at 40 psig, while the mixed-phase reaction was operated at 90 psig. Again, it was clearly demonstrated that operating under mixed-phase conditions resulted in an increase in total butenes conversion from approximately 62% up to 67%. It can also be seen that the mixed-phase and gas phase reactions follow similar deactivation curves.

While not intending to be limited to an interpretation of this phenomenon, we hypothesize that the operating pressure causes heavier components to preferentially condense on the catalyst or in the pores of the catalyst, while the lighter components do not tend to condense, or condense to a lesser extent.

Regeneration of Re₂O₇/Al₂O₃ Catalysts

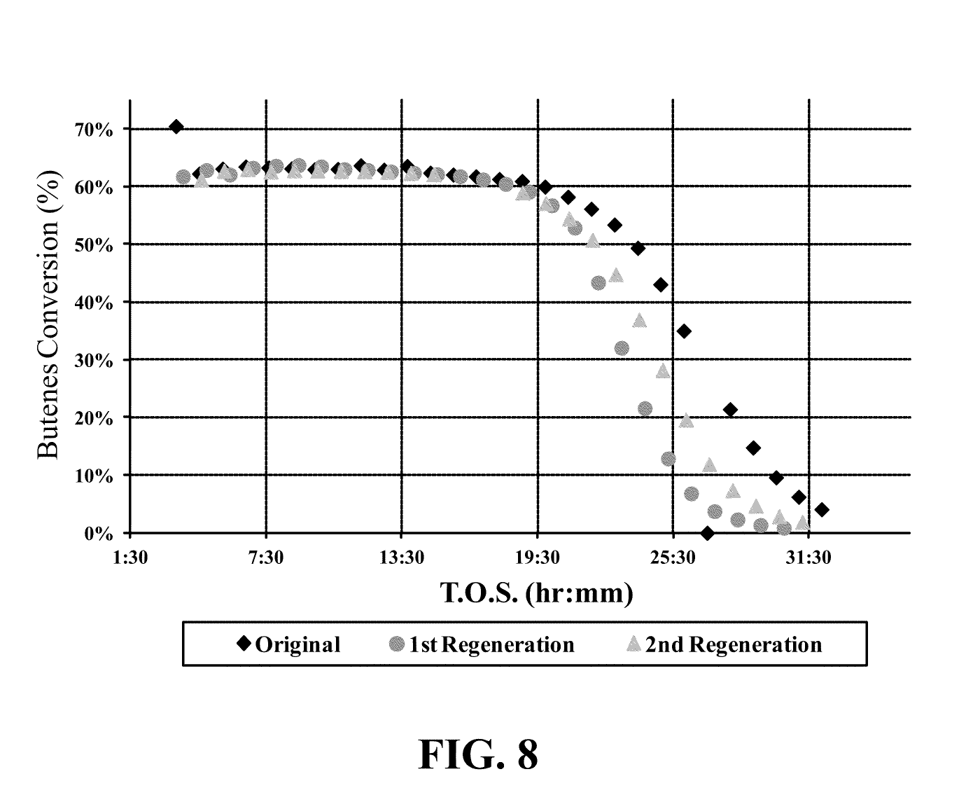

A 5 wt % Re₂O₇/Al₂O₃ catalyst was tested in the liquid phase at 100° C. to determine a baseline deactivation behavior. The diamond data points in FIG. 8 showed that the catalyst retains its equilibrium conversion value of 62% for nearly 14 hours time on stream; followed by complete deactivation by approximately 30 hours total time on stream. The catalyst was then subjected to an in-situ oxidation at 500° C. This was followed by re-exposure to metathesis reaction conditions and a complete deactivation curve was again determined. It can be seen from circle data points in FIG. 8 that the catalyst regains 100% of its original activity, but deactivated a little sooner than the fresh catalyst. After a second deactivation, the catalyst was regenerated a second time, and again the catalyst was found to reach 100% of its original activity as shown by the triangular data points in FIG. 8. This data clearly shows that the catalyst can be efficiently and effectively regenerated repeated times without loss in initial catalyst activity.

The data demonstrate that operating in the mixed-phase conditions has benefits including: (1) increased catalyst lifetime over the liquid phase and (2) increased butenes conversion, when operating above the dew point pressure so that the reaction mixtures is in a mixed-phase conditions. Thus, by careful manipulation of the operating conditions to a reaction temperature and a reaction pressures, where the reaction mixtures exists as a mixed-phase, gas and liquid, the metathesis reaction can be tuned to maximize propylene and 3-hexene production in the case of butenes feedstock. The data also demonstrate that the catalyst is regenerable in air at 500° C. after being completely deactivated under reaction conditions indicating that the mixed-phase reaction conditions are not deleterious to catalyst initial lifetime and catalyst regeneration lifetime.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A method for olefin metathesis comprising the step of:
contacting an olefin feed stream with a metathesis catalyst in a metathesis reactor at a reaction temperature and at a reaction pressure sufficient to maintain an olefin reaction mixture in a mixed-phase condition including components in the liquid phase and components in the gas phase to produce an olefin effluent stream; and
recovering an olefin product from the olefin effluent stream.

2. The method of claim 1, further comprising the steps of
fractionating the effluent stream to form a plurality of fraction streams; and,
recycling at least a portion of at least one of the plurality of fraction streams to the metathesis reactor;
wherein the step of recovering an olefin product from the olefin effluent stream comprises recovering an olefin product from at least a portion of at least one of the plurality of fraction streams.

3. The method of claim 2, further comprising the steps of directing at least a portion of at least one of the plurality of fraction streams to an olefin isomerization reactor and therein subjecting the same to olefin isomerization reaction.

4. The method of claim 2, further comprising the steps of directing at least a portion of at least one of the plurality of fraction streams to an aromatization reactor and therein subjecting the same to an aromitzation reaction.

5. The method of claim 2, further comprising the steps of directing the olefin feed stream to an olefin isomerization reactor and therein subjecting the same to an olefin isomerization reaction prior to the step of contacting.

6. The method of claim 1, wherein the reaction pressure is a pressure above a dew point pressure of the reaction mixture at the reaction temperature.

7. The method of claim 1, wherein the reaction pressure is above a dew point pressure and below a bubble point pressure of the reaction mixture at the reaction temperature.

8. The method of claim 1, wherein the reaction temperature and the reaction pressure are selected such that the olefin reaction mixture exists as a two phase system, a liquid phase and a gas phase, where components of the olefin reaction mixture are in the liquid phase and components of the olefin reaction mixture are in the gas phase.

9. The method of claim 8, wherein the reaction pressure is above a dew point pressure of at least one of the heavier olefins in the olefin reaction mixture at the reaction temperature.

10. The method of claim 1, wherein the reaction temperature is between about 40° and about 100° C. and the reaction pressure is sufficient to maintain the olefin reaction mixture in a mixed- phase region of the olefin reaction mixture phase diagram.

11. The method of claim 1, wherein the reaction temperature is between about 40° and about 90° C. and the reaction pressure is sufficient to maintain the olefin reaction mixture in a mixed-phase region of the olefin reaction mixture phase diagram.

12. The method of claim 1, wherein the reaction temperature is between about 40° and about 80° C. and the reaction pressure is sufficient to maintain the olefin reaction mixture in a mixed-phase region of the olefin reaction mixture phase diagram.

13. The method of claim 1, wherein the reaction temperature is between about 40° and about 60° C. and the reaction pressure is sufficient to maintain the olefin reaction mixture in a mixed-phase region of the olefin reaction mixture phase diagram.

14. The method of claim 1, wherein the starting olefin stream comprises a mixture of C₂ to C₁₂ olefins.

15. The method of claim 1, wherein the starting olefin stream comprises ethylene and butenes.

16. The method of claim 1, wherein the starting olefin stream comprises ethylene and pentenes.

17. The method of claim 1, wherein the starting olefin stream is a butenes stream including 1-butene and 2-butenes.

18. The method of claim 1, wherein the reaction pressure is above a dew point pressure of an the reaction mixture including ethylene, propylene, normal butenes, 2-pentene, and 3-hexene.

19. The method of claim 1, wherein the reaction pressure is above a dew point of at least 3-hexene.

20. The method of claim 1, wherein the metathesis catalyst comprises a catalyst capable of olefin metathesis.

21. The method of claim 1, wherein the metathesis catalyst is selected from the group consisting of supported and unsupported molybdenum metathesis catalysts, tungsten metathesis catalysts, rhenium metathesis catalysts, niobium metathesis catalysts, tantalum metathesis catalysts, tellurium metathesis catalysts, and mixtures or combinations thereof.

22. The method of claim 1, wherein the metathesis catalyst is selected from the group consisting of supported molybdenum metathesis catalysts, supported tungsten metathesis catalysts, supported rhenium metathesis catalysts, supported niobium metathesis catalysts, supported tantalum metathesis catalysts, supported tellurium oxide metathesis catalysts, supported molybdenum and tungsten sulfide metathesis catalysts, supported molybdenum and tungsten hexacarbonyl metathesis catalysts, and mixtures or combinations thereof.

23. The method of claim 1, wherein the metathesis catalyst comprises a rhenium metathesis catalyst.

24. The method of claim 1, wherein the metathesis catalyst comprises supported rhenium metathesis catalysts.

25. The method of claim 22, wherein the supported rhenium metathesis catalyst comprises Re₂O₇ and Al₂O₃.
